(19) United States
(12) Patent Application Publication
(10) Pub. No.: US 2026/0097189 A1
(43) Pub. Date: Apr. 9, 2026

(54) NAVIGATION DEVICE FOR USE WITH A FUNNEL CATHETER MINIMIZING CATCHING AT A VESSEL BIFURCATION

(71) Applicant: Neuravi Limited, Galway (IE)

(72) Inventors: Declan LEE, Longford (IE); Aidan DUFFY, Galway (IE)

(73) Assignee: Neuravi Limited, Galway (IE)

(21) Appl. No.: 18/910,053

(22) Filed: Oct. 9, 2024

(51) Int. Cl. A61M 25/10; A61B 17/22; A61M 25/00; A61M 25/01; A61M 25/09

(52) U.S. Cl. CPC A61M 25/10; A61B 17/22; A61M 25/0023; A61M 25/005; A61M 25/0147; A61M 25/09; A61B 2017/22038; A61B 2017/22071; A61M 2025/1045

(57) ABSTRACT

Endovascular medical system and method for preventing catching of a free terminating distal edge of a funnel catheter at a bifurcated vessel using a navigation device advanceable in a lumen of the funnel catheter and having a radially expandable section interposed between a tapered distal section and a proximal section. The radially expandable section of the navigation device in a radially expanded state has a maximum outer diameter substantially equal to the maximum inner diameter at the free terminating distal edge of the radially expandable distal section of the funnel catheter while in the radially expanded state. While traversing the bifurcated vessel, the radially expandable section of the navigation device in the radially expanded state prevents catching of the free terminating distal edge of the funnel catheter. Thereafter, the navigation device is collapsed and withdrawn from the funnel catheter.

### FIELD

The present disclosure relates to a navigation device for use with a funnel catheter herein defined as a catheter having a radially expandable distal section with a tapered outer contour, shape or profile. In particular, the present disclosure is directed to the navigation device having a radially expandable section filling or occluding the radially expandable distal section of the funnel catheter preventing catching on a vessel bifurcation while navigating through the vasculature to the target site (e.g., proximal side of a target clot to be captured).

### BACKGROUND

Catheters are widely used during a variety of endovascular medical treatments or procedures. In one such treatment catheters are used for endovascular capture and removal of a clot, occlusion or blockage in the vessel. During such treatment a catheter, preferably a funnel catheter having a radially expandable distal section with a tapered outer contour, profile or shape including the free terminating distal edge is advanced through the vasculature to a proximal side of the target clot, occlusion or blockage. Aspiration, a stentriever or both may be used to capture and remove the target clot, occlusion or blockage via the lumen of the catheter. The tapered shape and radially expandable distal section of the funnel catheter fosters capture of the entire target clot, occlusion or blockage. When advanced through tortuous vasculature using only conventional navigation devices (e.g., guide catheters/sheaths and/or guidewires), the distal edge of the radially expandable distal section of the funnel catheter while in a radially expanded state potentially risks getting caught on a vessel bifurcation hampering, or possibly preventing altogether, further advancement distally to the target site.

It is therefore desirable to develop an improved endovascular medical system including a funnel catheter having a radially expandable distal section together with a navigation device that fills, block or occludes thereby preventing or minimizing potential risk of the radially expandable distal section of the funnel catheter becoming caught on a vessel bifurcation.

### SUMMARY

An aspect of the present disclosure relates to an improved endovascular medical system including a funnel catheter having a radially expandable distal section together with a navigation device that prevents, or minimizes, potential risk of the radially expandable distal section of the funnel catheter becoming caught on a vessel bifurcation improving deliverability and usability.

Another aspect of the present disclosure is directed to a navigation device (i.e., tool) for use with a funnel catheter having a radially expandable distal section with a tapered outer contour, profile or shape, wherein the navigation device has a corresponding radially expandable section that while in a radially expanded state substantially, if not completely, fills (i.e., occludes or blocks) the free terminating distal edge of the radially expandable distal section of the funnel catheter preventing, or at least minimizing, potential risk of catching while traversing through the tortuous vasculature in a distal direction past (i.e., beyond) a vessel bifurcation.

Still another aspect of the present disclosure relates to an improved endovascular medical system including a navigation device (i.e., tool) for use with a funnel catheter having a radially expandable distal section with a tapered outer contour, profile or shape, wherein the navigation device has a corresponding radially expandable section that while in a radially expanded state has a maximum outer diameter substantially equal to an inner diameter at the free terminating distal edge of the radially expandable distal section of the funnel catheter while in the radially expanded state preventing, if not minimizing, potential risk of catching while traversing a vessel bifurcation during navigation through the tortuous vasculature to the target site.

### BRIEF DESCRIPTION OF THE DRAWINGS

The above and further aspects of the present disclosure are further discussed with reference to the following description in conjunction with the accompanying drawings, in which like numerals indicate like structural elements and features in various figures. The drawings are not necessarily to scale, emphasis instead being placed upon illustrating principles of the present disclosure. The figures depict one or more implementations of the devices of the present disclosure, by way of example only, not by way of limitation.

FIG. 1A is a side view of an example of the navigation device in accordance with the present disclosure having a radially expandable section interposed between a proximal section and a tapered distal section; wherein the radially expandable section of the navigation device includes a compliant polymer outer jacket covering a radially expandable mechanical structure (e.g., coil) activated by a pull wire attached thereto, the pull wire is illustrated in a deactivated (i.e., non-activated or advanced) state with the radially expandable section of the navigation device having a radially constricted outer diameter;

FIG. 1B is a side view of the example navigation device of FIG. 1A illustrated with the pull wire in an activated (i.e., retracted) state and the radially expandable section of the navigation device having a radially enlarged outer diameter;

FIG. 2A is a side view of another example of the navigation device in accordance with the present disclosure having a radially expandable section interposed between a proximal section and a tapered distal section; wherein the radially expandable section of the navigation device is a compliant polymer outer jacket of substantially uniform thickness in a longitudinal/axial direction defining an interior cavity fillable with a controllable volume of inflation medium (e.g., contrast medium and/or saline) and illustrated in a deactivated (i.e., non-activated) state having a radially constricted outer diameter;

FIG. 2B is a side view of the example navigation device of FIG. 2A illustrated in an activated state with the radially expandable section of the navigation device having a radially enlarged outer diameter;

FIG. 3A is a side view of yet another example of the navigation device in accordance with the present disclosure having a radially expandable section interposed between a proximal section and a tapered distal section; wherein the radially expandable section of the navigation device is a compliant polymer outer jacket of non-uniform thickness in a longitudinal/axial direction defining an interior cavity (free from any radially expanding mechanical structure) and a pull wire secured at a distal end to the assembled tapered distal section and guidewire; the radially expandable distal section of the navigation device is illustrated in a deactivated (i.e., non-activated) state having a radially constricted outer diameter with the pull wire in an advanced (i.e., non-retracted) state;

FIG. 3B is a side view of the example navigation device of FIG. 3A illustrated in an activated state with the pull wire in a retracted state (i.e., pulled in the proximal direction) and the radially expandable section of the navigation device having a radially enlarged outer diameter;

FIG. 4A is a schematic diagram of the problem addressed by the present disclosure wherein while navigating through the vasculature using only a conventional stand alone guidewire or guide catheter/sheath depicting the funnel catheter with the radially expandable distal section in the radially enlarged state catching or snagging at a vessel bifurcation hampering or preventing altogether advancement to the target site (e.g., on the proximal side of the target clot, occlusion or blockage);

FIG. 4B is a schematic diagram of the funnel catheter with the free terminating distal edge of the radially expandable distal section in the radially enlarged state is positioned in the vasculature on the proximal side of the vessel bifurcation and the navigation device (representing, but not limited to, any of the example navigation devices described or illustrated herein) with its radially expandable section in a radially enlarged state substantially aligned in the longitudinal/axial direction with the radially expandable distal section of the funnel catheter and substantially filling (i.e., occluding or blocking) the inner diameter at the free terminating distal edge of the funnel catheter preventing risk of catching while traversing the vessel bifurcation;

FIG. 4C is a schematic diagram of the funnel catheter with the radially expandable distal section in the radially enlarged state after traversing (i.e., advanced distally beyond) the vessel bifurcation and subsequent withdraw in the proximal direction of the navigation device from the funnel catheter allowing unhampered further advancement in a distal direction through the vasculature to the proximal side and capture of the clot, occlusion or blockage; and

FIG. 5 is a flow chart of the method of using an endovascular medical system in accordance with the present disclosure preventing, or minimizing, potential risk of a deployed funnel catheter catching at a vessel bifurcation by filling the free terminating distal edge of the radially expandable distal section of the deployed funnel catheter with a radially expandable section of a navigation device receivable therein.

### DETAILED DESCRIPTION

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “tubular” and “tube” are to be construed broadly and are not limited to a structure that is a right cylinder or strictly circumferential in cross-section or of a uniform cross-section throughout its length. For example, a tubular structure or system is generally illustrated as a substantially right cylindrical structure. However, the tubular system may have a tapered or curved outer surface without departing from the scope of the present disclosure.

Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

The present disclosure is directed to an endovascular medial system including a funnel catheter having a radially expandable distal section having a free terminating distal edge. A guide catheter or guide sheath is tracked through the tortuous vasculature. The funnel catheter is then advanced through the lumen of the microcatheter while the radially expandable distal section is in a radially constrained state. Upon exiting from the distal end of the guide catheter or guide sheath the radially expandable distal section of the funnel catheter transitions (i.e., deployed) to a radially enlarged state. While traversing the vasculature the free terminating distal edge of the radially expandable distal section of the funnel catheter may become caught on a vessel bifurcation hampering, or preventing altogether, further movement in a distal direction to a target site to be treated (e.g., capture of a target clot, occlusion or blockage distally of the vessel bifurcation). The navigation device (i.e., tool) in accordance with the present disclosure together with the funnel catheter as part of an endovascular medical system prevents catching or snagging at a vessel bifurcation of the free terminating distal edge of the deployed funnel catheter while traversing the vasculature to the target site.

Illustrated and described herein are several non-limiting exemplary configurations of the navigation device in accordance with the present disclosure. Other exemplary configurations of the navigation device are contemplated that include a proximal section, a tapered distal section (having a proximal end with a maximum outer diameter and an opposite distal end with a minimum outer diameter) and a radially expandable section interposed between the proximal section and the tapered distal section. The radially expandable section is transitionable between a radially constricted state having a radially constricted outer diameter and a radially expanded state having a radially enlarged outer diameter. Numerous ways (e.g., mechanically, fluidically, etc.) of activating or transitioning the radially expandable section of the navigation device are contemplated, not limited to those examples described and illustrated.

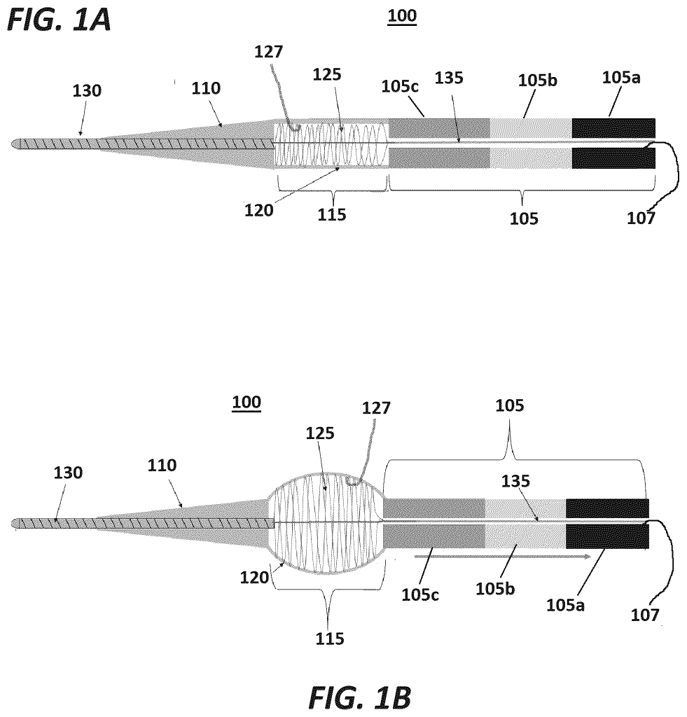

FIGS. 1A & 1B are schematic diagrams of a first exemplary navigation device 100 in accordance with the present disclosure depicted in a deactivated (i.e., non-activated or default) state (e.g., the radially expandable section of the navigation device in a radially constricted state) and an activated state (e.g., the radially expandable section of the navigation device in a radially enlarged or expanded state), respectively. Navigation device 100 includes a proximal section 105 made of at least one polymer jacket (e.g., a single polymer jacket of uniform durometer (i.e., stiffness) or a plurality of polymer jackets arranged serially one after the other abutting each other in the longitudinal/axial direction and of varying or different durometer (i.e., stiffness). Preferably, the stiffness is greatest at the proximal section 105a and lowest at the distal section 105c to maximize pushability at the proximal end while advancing through the guide catheter/sheath while optimizing flexibility at the distal end to navigate through the tortuous vasculature. Proximal section 105 may be a single jacket (i.e., outer layer) made of a single material of uniform durometer (i.e., stiffness). Alternatively, proximal section 105 may comprise multiple jackets arranged serially one after the other in the longitudinal direction abutting one another, wherein each jacket is made of a different material having a different durometer or stiffness. Proximal section is hereinafter generically referred to by reference element 105 representing any number of one of more jacket layers (105a, 105b, 105c, . . . , 105n). By way of illustrative example only, proximal section 105 throughout the figures includes three different jackets 105a, 105b, 105c arranged in series one abutting the other in the longitudinal/axial direction and each varying in durometer (i.e., stiffness). The stiffness is greatest at the proximal section 105a and lowest at the distal section 105c to maximize pushability and flexibility while traversing the tortuous pathway. Defined longitudinal/axially through the proximal section 105 is a lumen 107 having a pull wire or fiber 135 extending therethrough.

The tapered distal section 110 has a maximum outer diameter at the proximal end and a minimum outer diameter at the distal end. To optimize flexibility while tracking through the tortuous vasculature to a target site the tapered distal section is made of a flexible material such as polyurethane, polyethylene terephthalate (PET), polyamide, polyethylene and polyimide. Tapering of the distal section 110 in a distal direction is advantageously atraumatic. A guidewire 130 is permanently (i.e., fixedly or non-releasably) secured within and extends longitudinally/axially through the tapered distal section 110 projecting distally outward from (i.e., beyond) relative to the distal tip (of minimum outer diameter) of the tapered distal section 110. Since the guidewire 130 and tapered distal section 110 are permanently secured together acting as a single unitary piece, the guidewire 130 never moves and is never withdrawn relative to the tapered distal section 110. Accordingly, a distal portion of the guidewire 130 including the distal tip is free from, (i.e., not covered by the tapered distal section 110) to assist in navigation. However, a lubricious coating (e.g., hydrophilic coating) may be applied to the entire navigation device 100 from the proximal end to the distal end including the guide guidewire 130, or alternatively only a portion(s) thereof. The guidewire 130 is preferably made of a shapeable radiopaque material (e.g., platinum) having an outer diameter of approximately 0.014′. As shown the guidewire 130 is linear or straight, but may otherwise be pre-formed to have a non-linear shape (e.g., atraumatic shape such as J-shaped) distal end for navigating through the vessel. Spanning between the proximal section 105 and the tapered distal section 110 is a compliant outer polymer jacket 120 (e.g., elastomeric urethane, nylon, polyethylene, polyurethane, or polyolefin). The material selected for the outer polymer jacket 120 is sufficiently compliant to radially expand/enlarge when the navigation device is in the activated state, but sufficiently stiff/rigid when radially contracted/constricted to maintain pushability while the navigation device is in the deactivated state. A proximal edge of the outer polymer jacket 120 is permanently (i.e., fixedly or non-releasably) secured (e.g., via adhesive, weld, or mechanical securement such as via shrink wrap or a crimped marker band) to the proximal section 105, while the distal edge of the outer polymer jacket 120 is permanently (i.e., fixedly or non-releasably) secured (e.g., via adhesive, weld, or mechanical securement such as via shrink wrap or a crimped marker band) to the tapered distal section 110. An interior cavity or space 127 is defined bounded by the outer polymer jacket 120, the proximal section 105 and the tapered distal section 110. Disposed within the interior cavity 127 is a radially expandable mechanical structure 125 (e.g., spring, coil, stent, skeleton, frame or cage). Preferably, the radially expandable mechanical structure 125 is made of stainless steel, Nitinol or similar materials. Mechanical structure 125 is radially expandable in response to a mechanical activation mechanism 135. In FIGS. 1A & 1B spring 125 is activated by retracting (i.e., pulling in a proximal direction on a proximal end) a pull wire 135 passing longitudinally/axially freely through (i.e., without being attached to) the spring and permanently (i.e., fixedly or non-releasably) secured (e.g., via adhesive or weld) at a distal end to the proximal end of the guidewire 130 or tapered distal section 110. Referring to FIG. 1B, in response to pulling in the proximal direction (i.e., retracting) the pull wire 135, the spring 125 together with the compliant polymer outer jacket 120 covering the spring 125 expands radially in outer diameter (e.g., maximum outer diameter). Whereas the opposite movement, namely, advancement in a distal direction (i.e., release or cessation of pulling in the proximal direction), of the pull wire 135 causes the spring 125 together with the compliant polymer outer jacket 120 to automatically self-transition or revert to the radially constricted state (i.e., pre-formed original, natural or default state of minimum outer diameter).

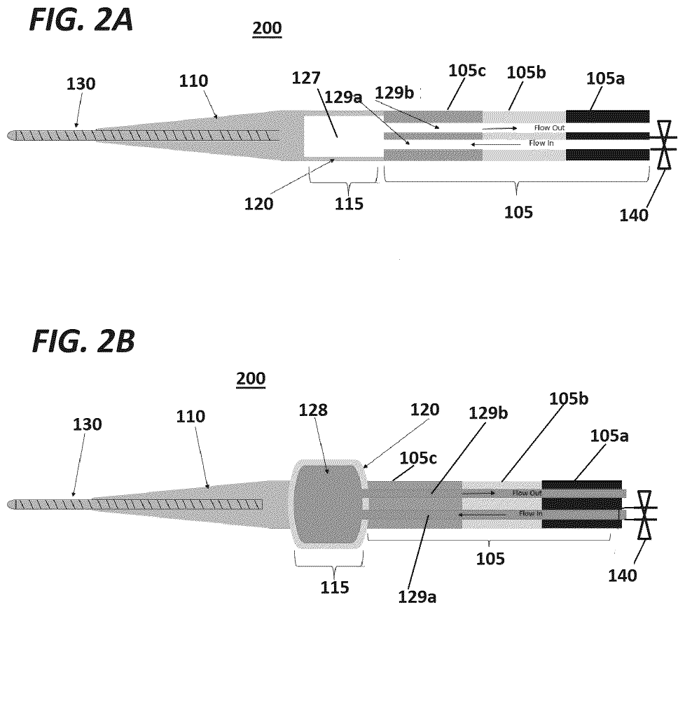

In a second exemplary embodiment depicted in FIGS. 2A & 2B the radially expandable section 115 of the navigation device 200 is fluidically activated (i.e., transitioned to a radially expanded or enlarged state) via injection of an inflation medium 128 (e.g., contrast medium and/or saline). By eliminating the mechanically radially expandable structure 125, the radially expandable section 115 of the navigation device 200 (FIGS. 2A & 2B) is more atraumatic than the navigation device 100 (FIGS. 1A & 1B). As with the example of FIGS. 1A & 1B, the compliant polymer outer jacket 120 in FIGS. 2A & 2B spans between the proximal section 105 and tapered distal section 110 defining therein an interior cavity 127. In FIGS. 2A & 2B the compliant polymer outer jacket 120 is permanently (i.e., fixedly or non-releasably) secured to the respective proximal section 105 and tapered distal section 110. However, rather than housing in the interior cavity 127 a radially expandable mechanical structure as in FIGS. 1A & 1B, in FIGS. 2A & 2B transitioning of the outer diameter between a radially expanded state and a radially constricted state is achieved exclusively fluidically by controlling a volume of inflation medium 128 (e.g., contrast medium and/or saline) injected into or purged from the interior cavity 127. Flow of the inflation medium 128 is controlled via one or more lumen in fluid communication with the interior cavity 127 and defined longitudinally/axially along the proximal section 105. Proximal section 105 in FIGS. 2A & 2B has defined therein two separate lumen 129a, 129b, arranged (e.g., concentrically, eccentrically or parallel side-by-side) to one another. Inlet lumen (i.e., inflation lumen) 129a is restricted to unidirectional flow of the injectable inflation medium 128 into the interior cavity 127 thus inflating (i.e., radially expanding) the compliant polymer outer jacket 120. While outlet lumen (i.e., deflation lumen) 129b is dedicated to unidirectional flow of the purged inflation medium 128 from the interior cavity 127 thus deflating (i.e., radially contracting) the compliant polymer outer jacket 120. Prepping or purging of the interior cavity 127 is realized by injecting inflation medium 128 (e.g., contrast saline solution) via inlet lumen 129a pushing existing air present in the interior cavity 127 via the outlet lumen 129b, until the injected inflation medium 128 exits from the outlet lumen 129b. Once purged of the existing air, the outlet lumen 129b is closed (i.e., shut via an associated valve 140), allowing for inflation (i.e., radial expansion) of the compliant outer jacket 120 via injection of additional inflation medium through inlet lumen 129a into the interior cavity 127 (FIG. 2B). Use of dual lumen reduces prep time, nevertheless, the use of a single lumen is also contemplated.

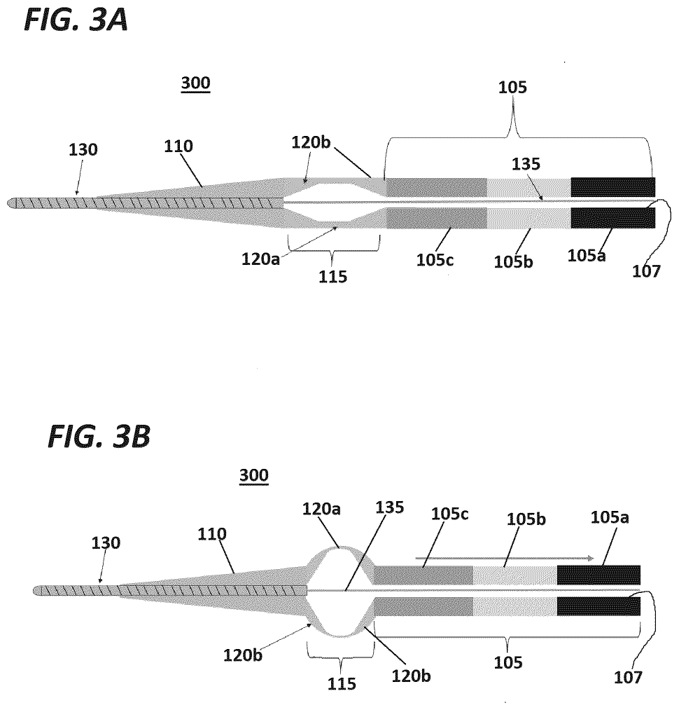

Still another example navigation device 300 is shown in FIGS. 3A & 3B wherein the radially expandable section 120 is once again activated by a mechanical activation mechanism (e.g., pull wire 135), but the mechanically radially expandable structure 125 disposed in the interior cavity 127 in the example of FIGS. 1A & 1B is eliminated. By eliminating the mechanically radially expandable structure 125, the navigation device 300 (FIGS. 3A & 3B) is more atraumatic than the navigation device 100 (FIGS. 1A & 1B). In the example of FIGS. 3A & 3B the interior cavity 127 is free or devoid of any radially expandable mechanical structure, rather the only mechanical structure present in the internal cavity 127 is the pulling wire 135 extending longitudinally therethrough and permanently secured at the distal end to the proximal end of the guidewire 130 or tapered distal section 110. To achieve radial expansion, the compliant outer jacket 120 in this example is non-uniform in radial thickness in the longitudinal/axial direction. As with the example of FIGS. 2A & 2B, the compliant polymer outer jacket 120 in FIGS. 3A & 3B spans between the proximal section 105 and tapered distal section 110 defining therein an interior cavity 127. Also, the compliant polymer outer jacket 120 is permanently (i.e., fixedly or non-releasably) secured to the respective proximal section 105 and tapered distal section 110. The radial thickness of the compliant polymer outer jacket 120 is tapered and non-uniform at its respective proximal and distal sections 120b while along an intervening region 120a therebetween is thinnest and substantially uniform. Specifically, the compliant outer jacket is substantially uniform in a longitudinal/axial direction along the intervening region 120a in the illustrated example while the compliant outer jacket along the respective proximal and distal sections 120b are mirror images of one another tapered increasingly in radial thickness in opposing directions longitudinally/axially starting from the intervening region 120a. Other examples of non-unform thickness in a longitudinal direction of the compliant outer jacket are contemplated in accordance with the present disclosure. For example, the intervening region 120a of uniform thickness may be eliminated altogether whereby the non-uniform proximal and distal sections 120b are mirror images of one another abutting one another at a boundary, interface or transition in radial thickness. That is, starting at the boundary, interface or transition in radial thickness in opposing directions in the longitudinal/axial direction the radial thickness of the compliant polymer outer layer 120b increases. FIG. 3A illustrates the navigation device 300 with the radially expandable section 115 in a radially constricted state while the pull wire 135 is in a non-activated or default state (i.e., non-retracted state). In response to the interventionalist or physician pulling in the proximal direction (i.e., retracting) the pull wire 135 the compliant outer jacket of non-uniform thickness 120a, 120b radially expands to an activated state in which the radially expandable section 115 is radially enlarged (e.g., maximum outer diameter), as depicted in FIG. 3B.

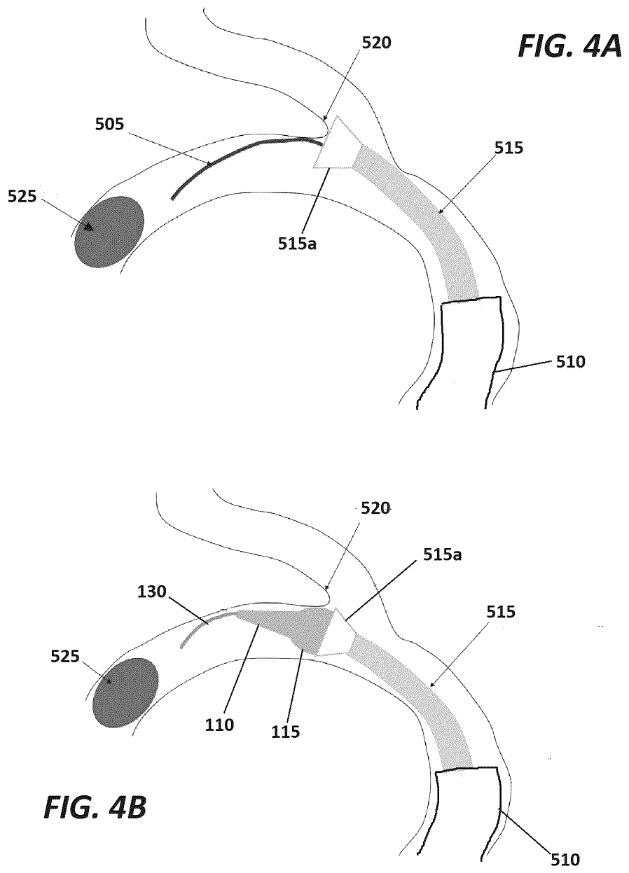

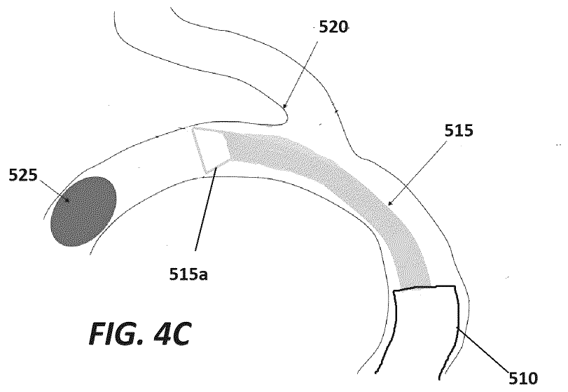

FIG. 4A schematically represents the problem addressed by the present disclosure wherein while navigating through the vasculature using only a conventional standalone guide catheter/sheath 510 and guidewire 505, the funnel catheter 515 with the radially expandable distal section 515a in the radially enlarged state potentially risks catching or snagging at a vessel bifurcation 520 hampering or preventing altogether advancement to the target site (e.g., on the proximal side of the target clot, occlusion or blockage 525).

In accordance with the present disclosure when encountering a vessel bifurcation 520 such risk of catching or snagging of the funnel catheter with the radially expandable distal section in the radially enlarged state is prevented. Specifically, while the funnel catheter 515 (with its radially expandable distal section 515a in a radially enlarged state) is positioned in the vasculature on the proximal side of the vessel bifurcation 520 the navigation device (representing, but not limited to, any of the example navigation devices described or illustrated herein) in accordance with the present disclosure while its radially expandable section 115 is in a radially constrained state is advanced through the funnel catheter 515. When the radially expandable section 115 of the navigation device 100, 200, 300 is longitudinally/axially substantially aligned with the radially enlarged distal section 515a of the funnel catheter 515, the radially expandable section 115 is actuated or deployed transitioning to a radially enlarged state. As discussed previously, actuation of the radially expandable section 115 of the navigation device may be mechanically (e.g., retraction of a pull wire 135) or fluidically (e.g., filling the interior space 127 of the radially expandable section 115 with inflation medium). In an actuated or deployed radially enlarged state the radially expandable section 115 of the navigation device 100, 200, 300 substantially, preferably completely, fills (i.e., occludes or blocks) the inner diameter at the free terminating distal edge of the radially expandable distal section 515a of the funnel catheter 515 ensuring the funnel catheter 515 bypasses, traverses or travels past the vessel bifurcation 520 without catching or snagging. Preferably, the radially expandable section 115 of the navigation device 100, 200, 300 does not expand or enlarge radially greater than the inner diameter of the free terminating distal edge of the radially enlarged distal section 515a of the funnel catheter 515 to avoid potentially damaging the vessel. To ensure maximized filling, blocking or occluding the navigation device 100, 200, 300 is preferably positioned so that its radially expandable section 115 of maximum outer diameter is preferably substantially aligned (i.e., coincides) with the free terminating distal edge of the radially expandable distal section 515a of the funnel catheter 515. In addition, radiopaque markers may be provided at the proximal and distal ends of the radial expandable section 115 to maintain proper alignment and positioning. Assembled together the funnel catheter 515 and the navigation device 100, 200, 300 traverse (i.e., travel or advance distally beyond) the vessel bifurcation 520 preventing, or at least minimizing, any catching or snagging of the free terminating edge of the radially expandable distal section 515a of the funnel catheter 515 on the vessel bifurcation 520, as depicted in FIG. 4B.

FIG. 4C is a schematic diagram depicting the funnel catheter with the radially expandable distal section 515a thereof in the radially enlarged state after bypassing, traversing or traveling distally past or beyond the vessel bifurcation 520 after the withdrawal in the proximal direction of the navigation device 100, 200, 300. That is, once the funnel catheter 515 has cleared the vessel bifurcation 520, the radially expandable section 115 of the navigation device 100, 200, 300 is transitioned, reverted or returned to the radially contracted state of reduced outer diameter sufficient to be withdrawn proximally through the funnel catheter 515 and removed from the body. Now that the vessel bifurcation 520 has been cleared, further advancement in the distal direction through the vasculature to the target site (e.g., proximal side or face of the clot 525) alone or by itself of the funnel catheter 515 while the radially expandable distal section 515a remains in the radially enlarged state continues unhampered or unhindered. When positioned at the target site (e.g., on the proximal side of the clot 525) aspiration and/or a mechanical retrieval device (e.g., stentriever) may be employed to capture and remove the clot 525.

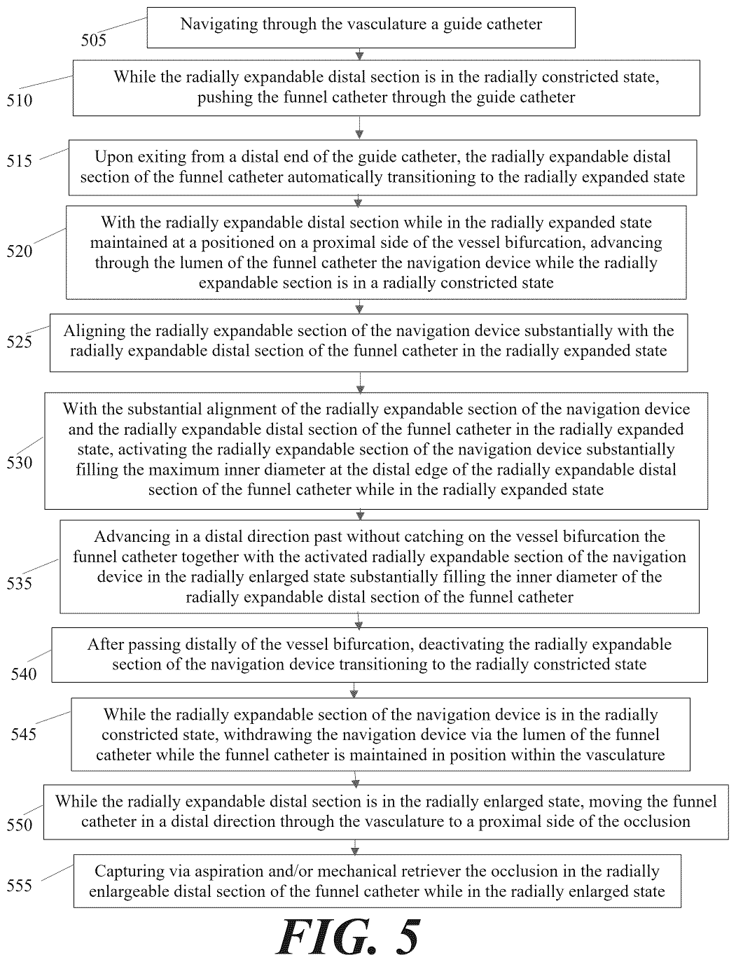

An exemplary flow chart of the method for using the navigation device (representing, but not limited to, any of the example navigation devices 100, 200, 300 illustrated and described herein) is provided in FIG. 5. In step 505 the guide catheter/sheath 510 is navigated through the vasculature (e.g., using a conventional standalone guidewire that is subsequently withdrawn). While the radially expandable distal section 515a is in the radially constricted state, in step 510 the funnel catheter 515 is pushed in a distal direction through the lumen of the guide catheter/sheath 510. Upon exiting from a distal end of the guide catheter/sheath 510, in step 515 the radially expandable distal section 515a of the funnel catheter 515 automatically transitions to the radially enlarged state. While maintaining at a position on a proximal side of the vessel bifurcation 120 the radially expandable distal section 515a of the funnel catheter 515 in the radially expanded/enlarged state, in step 520 the navigation device 100, 200, 300 with the radially expandable section 115 in a radially constricted state is advanced through the lumen of the funnel catheter 515. In step 525, the radially expandable section 115 of the navigation device 100, 200, 300 is substantially aligned in the longitudinal/axial direction with the radially expandable distal section 515a of the funnel catheter 515. With the substantial alignment of the radially expandable section 115 of the navigation device 100, 200, 300 and the radially expandable distal section 515a of the funnel catheter 515 in the radially expanded state, in step 530 the radially expandable section 115 of the navigation device 100, 200, 300 is activated/deployed substantially, preferably completely (i.e., fully, totally or entirely), filling (i.e., blocking or occluding) the maximum inner diameter at the free terminating distal edge of the radially expandable distal section 515a of the funnel catheter 515 while in the radially expanded state. Now in step 535, the funnel catheter 515 with the activated radially expandable section 115 of the navigation device 100, 200, 300 in the radially enlarged state substantially filling the inner diameter of the radially expandable distal section of the funnel catheter assembled together are advanceable in a distal direction through the vasculature bypassing, traversing or traveling past without catching on the vessel bifurcation 520. After passing distally of and thus clearing the vessel bifurcation 520, in step 540 the radially expandable section 115 of the navigation device 100, 200, 300 is deactivated transitioning to the radially constricted state (e.g., default state). While the radially expandable section 115 of the navigation device 100, 200, 300 is in the radially constricted state, in step 545 the navigation device 100, 200, 300 is withdrawn via the lumen of the funnel catheter 515 while the funnel catheter 515 itself is maintained in place within the vasculature. Now that the vessel bifurcation 520 is cleared and the risk of catching has been averted, while the radially expandable distal section 515a is in the radially enlarged state, the funnel catheter 515 is advanced further in the distal direction through the vasculature to the proximal side of the occlusion 525 (step 550) and then captured (via aspiration and/or stentriever) within the funnel catheter 515 for removal/withdraw.

It is noted that regardless of the particular configuration of the navigation device 100, 200, 300, the radially expandable section 115 is the only section or portion of the overall navigation device 100, 200, 300 to radially expand in outer diameter. Accordingly, the outer contour, shape or profile of the proximal section 105 and the tapered distal section 115 each remains constant, unchanging (i.e., non-radially expandable). As previously noted, any desired method or manner of activation of radial expansion of the radially expandable section 115 of the navigation device 100, 200, 300 transitioning between the radially constricted state and the radially enlarged state is possible.

Aspects of the present disclosure are also provided by the following numbered Clauses:

Clause 1: An endovascular medical system preventing catching at a bifurcated vessel (520) during removal of a clot (525); wherein the endovascular medical system comprises: a funnel catheter (515) having a proximal end and an opposite distal end defining a lumen therebetween; the funnel catheter (515) having a radially expandable distal section (515a) including a free terminating distal edge; the radially expandable distal section (515a) of the funnel catheter (515) being transitionable between a radially constricted state and a radially expanded state; and the radially expandable distal section (515a) of the funnel catheter (515) while in the radially expanded state has a maximum inner diameter at the free terminating distal edge; a navigation device (100, 200, 300) advanceable in the lumen of the funnel catheter (515); the navigation device (100, 200, 300) comprises: a tapered distal section (110) having a proximal end of maximum outer diameter and a distal end of minimum outer diameter; a proximal section (105); a radially expandable section (115) interposed between the tapered distal section (110) and the proximal section (105); the radially expandable section (115) of the navigation device (100, 200, 300) is transitionable between a radially constricted state and a radially expanded state; wherein in the radially constricted state, the radially expandable section (115) of the navigation device (100, 200, 300) has an outer diameter slidable within the lumen of the funnel catheter (515); and wherein in the radially expanded state, the radially expandable section (115) of the navigation device (100, 200, 300) has a maximum outer diameter substantially equal to the maximum inner diameter at the free terminating distal edge of the radially expandable distal section (515a) of the funnel catheter (515) while in the radially expanded state; and a guidewire (130) permanently secured to the tapered distal section (110), the guidewire (130) having a distal portion projecting distally in a longitudinal direction beyond the distal end of the tapered distal section (110).

Clause 2: The system of Clause 1, wherein the radially expandable section (115) of the navigation device (100) comprises: a compliant polymer outer jacket (120) secured between the proximal section (105) and the distal section (110) of the navigation device (100, 200, 300) defining an inner cavity (127); a radially expandable mechanical structure (125) disposed in the inner cavity (127); the radially expandable mechanical structure (125) is transitionable between a radially constricted state and a radially expanded state;
wherein transitioning of the radially expandable mechanical structure (125) between the radially constricted state and the radially expanded state is controllable via a mechanical activation device (135).

Clause 3: The system of Clause 2, wherein the mechanical activation device is a pull wire (135) secured at a distal end to either the guidewire (130) or the tapered distal section (110) of the navigation device (300).

Clause 4: The system of Clause 3, wherein the radially expandable mechanical structure (125) is a coil or braided structure.

Clause 5: The system of Clause 2, wherein the compliant polymer outer jacket (120) is substantially uniform in radial thickness in a longitudinal direction.

Clause 6: The system of Clause 1, wherein the radially expandable section (115) of the navigation device (200) comprises: a compliant polymer outer jacket (120) secured between the proximal (105) and distal sections (110) of the navigation device (200) defining an interior cavity (127); wherein the compliant polymer outer jacket (120) is transitionable from the radially constricted state to the radially expandable state by fluidically controlling a volume of inflation medium in the interior cavity (127); wherein the interior cavity (127) is in fluid communication with at least one lumen (129a, 129b) defined in the proximal section (105).

Clause 7: The system of Clause 6, wherein the at least one lumen comprises a single lumen (129a) in fluid communication with the interior cavity (127) for bi-directional flow of the inflation medium to and from the interior cavity (127).

Clause 8: The system of Clause 6, wherein the at least one lumen comprises: an inlet lumen (129a) for unidirectional flow of the contrast medium into the interior cavity (127); and an outlet lumen (129b) for unidirectional flow of the contrast medium from the interior cavity (127).

Clause 9: The system of Clause 1, wherein the radially expandable section (115) of the navigation device (300) comprises: a compliant polymer outer jacket (120) secured between the proximal section (105) and the distal section (110) of the navigation device (300) defining an interior cavity (127);
wherein the compliant polymer outer jacket (120) has a non-uniform wall thickness (120a, 120b) in a longitudinal direction; and the compliant polymer outer jacket (120) is transitionable from the radially constricted state to the radially expandable state via a mechanical activation device (135).

Clause 10: The system of Clause 9, wherein the mechanical activation device (135) is a pull wire having a proximal end and an opposite distal end; the pull wire (135) extending longitudinally through the interior cavity (127) of the compliant polymer outer layer (120) with the distal end of the pull wire (135) secured to either the guidewire (130) or the tapered distal section (110) of the navigation device (300).

Clause 11: A method for using an endovascular medical system to prevent catching at a vessel bifurcation (520) during removal of a clot (525); wherein the endovascular medical system comprises: a funnel catheter (515) having a proximal end and an opposite distal end defining a lumen there between; the funnel catheter (515) having a radially expandable distal section (515a) including a distal edge; the radially expandable distal section (515a) of the funnel catheter (515) being transitionable between a radially constricted state and a radially expanded state; and the radially expandable distal section (515a) of the funnel catheter (515) while in the radially expanded state has a maximum inner diameter at the distal edge; and a navigation device (100, 200, 300) advanceable in the lumen of the funnel catheter (515); wherein the navigation device (100, 200, 300) comprises: a tapered distal section (110) having a proximal end of maximum outer diameter and a distal end of minimum outer diameter; a proximal section (105); a radially expandable section (115) interposed between the tapered distal section (110) and the proximal section (105); the radially expandable section (115) of the navigation device (100, 200, 300) is transitionable between a radially constricted state and a radially expanded state; wherein in the radially constricted state, the radially expandable section (115) of the navigation device (100, 200, 300) has an outer diameter slidable within the lumen of the funnel catheter (515); and wherein in the radially expanded state, the radially expandable section (115) of the navigation device (100, 200, 300) has a maximum outer diameter substantially equal to the maximum inner diameter at the distal edge of the radially expandable distal section (515a) of the funnel catheter (515) while in the radially expanded state; the navigation device further including a guidewire (130) permanently secured to the tapered distal section (110), the guidewire (130) having a distal portion projecting distally in a longitudinal direction beyond the distal end of the tapered distal section (110); the method comprising the steps of: navigating through the vasculature a guide catheter (510); while the radially expandable distal section (515a) is in the radially constricted state, pushing the funnel catheter (515) through the guide catheter (510); upon exiting from a distal end of the guide catheter (510), the radially expandable distal section (515a) of the funnel catheter (515) automatically transitioning to the radially expanded state; with the radially expandable distal section (515a) of the funnel catheter (515) while in the radially expanded state maintained at a positioned on a proximal side of the vessel bifurcation (520), advancing through the lumen of the funnel catheter (515) the navigation device (100, 200, 300) while the radially expandable section (115) of the navigation device (100, 200, 300) is in a radially constricted state; substantially aligning the radially expandable section (115) of the navigation device (100, 200, 300) with the radially expandable distal section (515a) of the funnel catheter (515) in the radially expanded state; with the substantial alignment of the radially expandable section (115) of the navigation device (100, 200, 300) and the radially expandable distal section (515a) of the funnel catheter (515) in the radially expanded state, activating the radially expandable section (115) of the navigation device (100, 200, 300) substantially filling the maximum inner diameter at the distal edge of the radially expandable distal section (515a) of the funnel catheter (515) while in the radially expanded state; advancing in a distal direction past without catching on the vessel bifurcation (520) the funnel catheter (515) together with the activated radially expandable section (115a) of the navigation device (100, 200, 300) in the radially enlarged state substantially filling the inner diameter of the radially expandable distal section (515a) of the funnel catheter (515); after passing distally of the vessel bifurcation (520), deactivating the radially expandable section (115) of the navigation device (100, 200, 300) transitioning to the radially constricted state; while the radially expandable section (115) of the navigation device (100, 200, 300) is in the radially constricted state, withdrawing the navigation device (100, 200, 300) via the lumen of the funnel catheter (515) while the funnel catheter (515) is maintained in position within the vasculature.

Clause 12: The method of Clause 11, further comprising the steps of: while the radially expandable distal section (515a) is in the radially enlarged state, moving the funnel catheter (515) in a distal direction through the vasculature to a proximal side of the clot (525); and capturing via aspiration the clot (525) in the radially enlargeable distal section (515a) of the funnel catheter (515) while in the radially enlarged state.

Clause 13: The method of Claus 11, wherein the radially expandable section (115) of the navigation device (100) comprises: a compliant polymer outer jacket (120) secured between the proximal section (105) and the tapered distal section (110) of the navigation device (100, 200, 300) defining an inner cavity (127); a radially expandable mechanical structure (125) disposed in the inner cavity (127); the radially expandable mechanical structure (125) is transitionable between a radially constricted state and a radially expanded state; wherein transitioning of the radially expandable mechanical structure (125) between the radially constricted state and the radially expanded state is controllable via a mechanical activation device (135).

Clause 14: The method of Clause 13, wherein the mechanical activation device is a pull wire (135) secured to either the guidewire (130) or the tapered distal section (110).

Clause 15: The method of Clause 14, wherein the radially expandable mechanical structure (125) is a coil or braided structure.

Clause 16: The method of Clause 13, wherein the compliant polymer outer jacket (120) is substantially uniform in thickness.

Clause 17: The method of Clause 11, wherein the radially expandable section (115) of the navigation device (200) comprises: a compliant polymer outer jacket (120) secured between the proximal (105) and distal sections (110) of the navigation device (200) defining an interior cavity (127); wherein the compliant polymer outer jacket (120) is transitionable from the radially constricted state to the radially expandable state by fluidically controlling a volume of inflation medium in the interior cavity (127); wherein the interior cavity (127) is in fluid communication with at least one lumen (129a, 129b) defined in the proximal section (105).

Clause 18: The method of Clause 17, wherein the at least one lumen comprises: (i) a single lumen (129a) in fluid communication with the interior cavity (127) for bi-directional flow of the inflation medium to and from the interior cavity (127); or (ii) an inlet lumen (129a) for unidirectional flow of the contrast medium into the interior cavity (127); and an outlet lumen (129b) for unidirectional flow of the contrast medium from the interior cavity (127).

Clause 19: The method of Clause 11, wherein the radially expandable section (115) of the navigation device (300) comprises: a compliant polymer outer jacket (120) secured between the proximal section (105) and the distal section (110) of the navigation device (300) defining an interior cavity (127); wherein the compliant polymer outer jacket (120) has a non-uniform wall thickness (120a, 120b) in an axial direction; and the compliant polymer outer jacket (120) is transitionable from the radially constricted state to the radially expandable state via a mechanical activation device (135).

Clause 20: The method of Clause 19, wherein the mechanical activation device (135) is a pull wire having a proximal end and an opposite distal end; the pull wire (135) extending longitudinally through the interior cavity (127) of the compliant polymer outer layer (120) with the distal end of the pull wire (135) secured to either the guidewire (130) or the tapered distal section (110) of the navigation device.

The descriptions contained herein are examples and not intended in any way to limit the scope of the present disclosure. As described herein, the present disclosure contemplates many variations and modifications of the navigation device for use with a funnel catheter having a radially expandable distal section to prevent risk of catching or snagging at a vessel bifurcation. The navigation device includes a proximal section, a tapered distal section, a radially expandable section therebetween and a guidewire permanently secured to the tapered distal section and projecting distally therefrom. When the navigation device is assembled within the funnel catheter and activated, the radially expandable section of the navigation device fills, blocks or occludes, substantially, preferably completely, the inner diameter of the free terminating distal edge of the radially expandable distal section of the funnel catheter while in the radially expanded/enlarged state. Modifications and variations apparent to those having skilled in the pertinent art according to the teachings of this disclosure are intended to be within the scope of the claims which follow.

What is claimed is:

1. An endovascular medical system preventing catching at a bifurcated vessel during removal of a clot; wherein the endovascular medical system comprises:
a funnel catheter having a proximal end and an opposite distal end defining a lumen therebetween; the funnel catheter having a radially expandable distal section including a free terminating distal edge; the radially expandable distal section of the funnel catheter being transitionable between a radially constricted state and a radially expanded state; and the radially expandable distal section of the funnel catheter while in the radially expanded state has a maximum inner diameter at the free terminating distal edge;
a navigation device advanceable in the lumen of the funnel catheter; the navigation device comprises:
a tapered distal section having a proximal end of maximum outer diameter and a distal end of minimum outer diameter;
a proximal section;
a radially expandable section interposed between the tapered distal section and the proximal section; the radially expandable section of the navigation device is transitionable between a radially constricted state and a radially expanded state; wherein in the radially constricted state, the radially expandable section of the navigation device has an outer diameter slidable within the lumen of the funnel catheter; and wherein in the radially expanded state, the radially expandable section of the navigation device has a maximum outer diameter substantially equal to the maximum inner diameter at the free terminating distal edge of the radially expandable distal section of the funnel catheter while in the radially expanded state; and
a guidewire permanently secured to the tapered distal section, the guidewire having a distal portion projecting distally in a longitudinal direction beyond the distal end of the tapered distal section.

2. The system in accordance with claim 1, wherein the radially expandable section of the navigation device comprises:
a compliant polymer outer jacket secured between the proximal section and the distal section of the navigation device defining an inner cavity;
a radially expandable mechanical structure disposed in the inner cavity; the radially expandable mechanical structure is transitionable between a radially constricted state and a radially expanded state;
wherein transitioning of the radially expandable mechanical structure between the radially constricted state and the radially expanded state is controllable via a mechanical activation device.

3. The system in accordance with claim 2, wherein the mechanical activation device is a pull wire secured at a distal end to either the guidewire or the tapered distal section of the navigation device.

4. The system in accordance with claim 3, wherein the radially expandable mechanical structure is a coil or braided structure.

5. The system in accordance with claim 2, wherein the compliant polymer outer jacket is substantially uniform in radial thickness in a longitudinal direction.

6. The system in accordance with claim 1, wherein the radially expandable section of the navigation device comprises:
a compliant polymer outer jacket secured between the proximal and distal sections of the navigation device defining an interior cavity; wherein the compliant polymer outer jacket is transitionable from the radially constricted state to the radially expandable state by fluidically controlling a volume of inflation medium in the interior cavity; wherein the interior cavity is in fluid communication with at least one lumen defined in the proximal section.

7. The system in accordance with claim 6, wherein the at least one lumen comprises a single lumen in fluid communication with the interior cavity for bi-directional flow of the inflation medium to and from the interior cavity.

8. The system in accordance with claim 6, wherein the at least one lumen comprises: an inlet lumen for unidirectional flow of the contrast medium into the interior cavity; and an outlet lumen for unidirectional flow of the contrast medium from the interior cavity.

9. The system in accordance with claim 1, wherein the radially expandable section of the navigation device comprises:
a compliant polymer outer jacket secured between the proximal section and the distal section of the navigation device defining an interior cavity; wherein the compliant polymer outer jacket has a non-uniform wall thickness in a longitudinal direction; and the compliant polymer outer jacket is transitionable from the radially constricted state to the radially expandable state via a mechanical activation device.

10. The system in accordance with claim 9, wherein the mechanical activation device is a pull wire having a proximal end and an opposite distal end; the pull wire extending longitudinally through the interior cavity of the compliant polymer outer layer with the distal end of the pull wire secured to either the guidewire or the tapered distal section of the navigation device.

11. A method for using an endovascular medical system to prevent catching at a vessel bifurcation during removal of a clot; wherein the endovascular medical system comprises: a funnel catheter having a proximal end and an opposite distal end defining a lumen therebetween; the funnel catheter having a radially expandable distal section including a distal edge; the radially expandable distal section of the funnel catheter being transitionable between a radially constricted state and a radially expanded state; and the radially expandable distal section of the funnel catheter while in the radially expanded state has a maximum inner diameter at the distal edge; and a navigation device advanceable in the lumen of the funnel catheter; wherein the navigation device comprises: a tapered distal section having a proximal end of maximum outer diameter and a distal end of minimum outer diameter; a proximal section; a radially expandable section interposed between the tapered distal section and the proximal section; the radially expandable section of the navigation device is transitionable between a radially constricted state and a radially expanded state; wherein in the radially constricted state, the radially expandable section of the navigation device has an outer diameter slidable within the lumen of the funnel catheter; and wherein in the radially expanded state, the radially expandable section of the navigation device has a maximum outer diameter substantially equal to the maximum inner diameter at the distal edge of the radially expandable distal section of the funnel catheter while in the radially expanded state; the navigation device further including a guidewire permanently secured to the tapered distal section, the guidewire having a distal portion projecting distally in a longitudinal direction beyond the distal end of the tapered distal section; the method comprising the steps of:
navigating through the vasculature a guide catheter;
while the radially expandable distal section is in the radially constricted state, pushing the funnel catheter through the guide catheter;
upon exiting from a distal end of the guide catheter, the radially expandable distal section of the funnel catheter automatically transitioning to the radially expanded state;
with the radially expandable distal section of the funnel catheter while in the radially expanded state maintained at a positioned on a proximal side of the vessel bifurcation, advancing through the lumen of the funnel catheter the navigation device while the radially expandable section of the navigation device is in a radially constricted state;
substantially aligning the radially expandable section of the navigation device with the radially expandable distal section of the funnel catheter in the radially expanded state;
with the substantial alignment of the radially expandable section of the navigation device and the radially expandable distal section of the funnel catheter in the radially expanded state, activating the radially expandable section of the navigation device substantially filling the maximum inner diameter at the distal edge of the radially expandable distal section of the funnel catheter while in the radially expanded state;
advancing in a distal direction past without catching on the vessel bifurcation the funnel catheter together with the activated radially expandable section of the navigation device in the radially enlarged state substantially filling the inner diameter of the radially expandable distal section of the funnel catheter;
after passing distally of the vessel bifurcation, deactivating the radially expandable section of the navigation device transitioning to the radially constricted state;
while the radially expandable section of the navigation device is in the radially constricted state, withdrawing the navigation device via the lumen of the funnel catheter while the funnel catheter is maintained in position within the vasculature.

12. The method in accordance with claim 11, further comprising the steps of:
while the radially expandable distal section is in the radially enlarged state, moving the funnel catheter in a distal direction through the vasculature to a proximal side of the clot; and
capturing via aspiration the clot in the radially enlargeable distal section of the funnel catheter while in the radially enlarged state.

13. The method in accordance with claim 11, wherein the radially expandable section of the navigation device comprises:
a compliant polymer outer jacket secured between the proximal section and the tapered distal section of the navigation device defining an inner cavity;
a radially expandable mechanical structure disposed in the inner cavity; the radially expandable mechanical structure is transitionable between a radially constricted state and a radially expanded state;
wherein transitioning of the radially expandable mechanical structure between the radially constricted state and the radially expanded state is controllable via a mechanical activation device.

14. The method in accordance with claim 13, wherein the mechanical activation device is a pull wire secured to either the guidewire or the tapered distal section.

15. The method in accordance with claim 14, wherein the radially expandable mechanical structure is a coil or braided structure.

16. The method in accordance with claim 13, wherein the compliant polymer outer jacket is substantially uniform in thickness.

17. The method in accordance with claim 11, wherein the radially expandable section of the navigation device comprises: a compliant polymer outer jacket secured between the proximal and distal sections of the navigation device defining an interior cavity; wherein the compliant polymer outer jacket is transitionable from the radially constricted state to the radially expandable state by fluidically controlling a volume of inflation medium in the interior cavity; wherein the interior cavity is in fluid communication with at least one lumen defined in the proximal section.

18. The method in accordance with claim 17, wherein the at least one lumen comprises: (i) a single lumen in fluid communication with the interior cavity for bi-directional flow of the inflation medium to and from the interior cavity; or (ii) an inlet lumen for unidirectional flow of the contrast medium into the interior cavity; and an outlet lumen for unidirectional flow of the contrast medium from the interior cavity.

19. The method in accordance with claim 11, wherein the radially expandable section of the navigation device comprises:
a compliant polymer outer jacket secured between the proximal section and the distal section of the navigation device defining an interior cavity; wherein the compliant polymer outer jacket has a non-uniform wall thickness in an axial direction; and the compliant polymer outer jacket is transitionable from the radially constricted state to the radially expandable state via a mechanical activation device.

20. The method in accordance with claim 19, wherein the mechanical activation device is a pull wire having a proximal end and an opposite distal end; the pull wire extending longitudinally through the interior cavity of the compliant polymer outer layer with the distal end of the pull wire secured to either the guidewire or the tapered distal section of the navigation device.
